(19) United States
(12) Patent Application Publication
(10) Pub. No.: US 2016/0060599 A1
(43) Pub. Date: Mar. 3, 2016

(54) DENDRITIC CELL TUMOR VACCINE AND METHOD FOR PREPARING THE SAME

(71) Applicant: China Medical University Hospital, Taichung City (TW)

(72) Inventors: Der-Yang CHO, Taichung City (TW); Shao-Chih CHIU, Taichung City (TW)

(21) Appl. No.: 14/595,220

(22) Filed: Jan. 13, 2015

(30) Foreign Application Priority Data
Aug. 29, 2014 (TW) 103129948

(51) Int. Cl. C12N 5/0784; A61K 39/00

(52) U.S. Cl. CPC C12N 5/0639; A61K 39/0011; A61K 2039/5154; C12N 2502/1121

(57) ABSTRACT

A method of preparing the dendritic cell tumor vaccine includes steps as follows. A tumor specimen is primarily isolated and cultured to obtain a plurality of tumor cells. Cancer stem cells having a specific cell surface marker are sorted from the tumor cells. The cancer stem cells are irradiated with a radiation. A plurality of dendritic cells are provided. The dendritic cells and the cancer stem cells irradiated with the radiation are co-cultured for activating the dendritic cells into cancer-stem-cell-antigen-presenting dendritic cells to obtain the dendritic cell tumor vaccine. The dendritic cell tumor vaccine is a mixture of the cancer-stem-cell-antigen-presenting dendritic cells and the cancer stem cells.

### RELATED APPLICATIONS

This application claims priority to Taiwan Application Serial Number 103129948, filed Aug. 29, 2014, which is herein incorporated by reference.

### BACKGROUND

1. Technical Field

The present disclosure relates to a tumor vaccine. More particularly, the present disclosure relates to a dendritic cell tumor vaccine for cancer stem cells.

2. Description of Related Art

The conventional practices for cancer treatments are a surgery, a radiation therapy and a chemotherapy. Alternatively, a tumor vaccine is one treatment for the cancer treatment except the aforementioned treatments. A tumor vaccine activates a patient's own immune system by using tumor cells or tumor antigen substances to induce the patient's specific cellular and humoral immune response, so that it can enhance the patient's anti-cancer ability and prevent growth, proliferation and recurrence of the tumor. As a result, the tumor can be removed or controlled.

A dendritic cell is an antigen--presenting cell, whose main function is to process antigens and present antigens on the cell surface thereof to T cells of the immune system. T cells can play immune effectors and destroy cells hence the immune system response can be initiated. A dendritic cell tumor vaccine, including the functional dendritic cells presenting tumor antigen information, can induce an antigen-specific T cell in response to tumor antigen in order to exert anti-tumor effect and immune memory.

The conventional dendritic cell tumor vaccines target to primary tumor cells by lysing primary tumor cells to obtain antigens, and then loading the antigens to the dendritic cells obtained from the same patient. However, it is hard to solve the problem of tumor recurrence by using the aforementioned dendritic cell tumor vaccines to treat cancer. The main reason is that the aforementioned dendritic cell tumor vaccines cannot destroy all cancer stem cells of tumor, so that cancerous tissues recurrently grow and a handful of the cancer stem cells can drive the tumor formation.

### SUMMARY

According to one aspect of the present disclosure, a method of preparing a dendritic cell tumor vaccine includes steps as follows. A tumor specimen is primarily isolated and cultured to obtain a plurality of tumor cells. Cancer stem cells having a specific cell surface marker are sorted from the tumor cells. The cancer stem cells are irradiated with a radiation. A plurality of dendritic cells are provided. The dendritic cells and the cancer stem cells irradiated with the radiation are co-cultured for activating the dendritic cells into cancer-stem-cell-antigen-presenting dendritic cells to obtain the dendritic cell tumor vaccine, wherein the dendritic cell tumor vaccine is a mixture of the cancer-stem-cell-antigen-presenting dendritic cells and the cancer stem cells.

According to another aspect of present disclosure, a dendritic cell tumor vaccine is provided, The dendritic cell tumor vaccine includes a plurality of cancer-stem-cell-antigen-presenting dendritic cells and a plurality of cancer stem cells, wherein the cancer stem cells are mixed with the cancer-stem-cell-antigen-presenting dendritic cells.

### BRIEF DESCRIPTION OF THE DRAWINGS

The present disclosure can be more fully understood by reading the following detailed description of the embodiment, with reference made to the accompanying drawings as follows:

FIG. 1 is a flow diagram showing a method of preparing a dendritic cell tumor vaccine according to one embodiment of the present disclosure;

FIG. 2 are micrographs of monolayer cell culture and neuro-sphere formation of GBM (glioblastoma multiforme) cells;

FIG. 3 is a bar chart illustrating relative expression ratio of mRNA of the GSCs (glioblastoma stem cells) to the GBM cells according to another embodiment of the present disclosure;

FIG. 4A is a micrograph of an intracranial xenograft mice model of the GSCs by using hematoxylin and eosin staining according to another embodiment of the present disclosure;

FIG. 4B is a partially enlarged view of B in FIG. 4A;

FIG. 4C is a micrograph of the intracranial xenograft mice model of the GSCs by using hematoxylin and eosin staining according to another embodiment of the present disclosure;

FIG. 4D is a partially enlarged view of D in FIG. 4C;

FIG. 5A is a set of histograms of FACS (fluorescence-activated cell sorting) analysis showing a cytotoxic effect for CD133 negative glioblastoma multiforme cells of a dendritic cell tumor vaccine according to a comparative example;

FIG. 5B is a set of histograms of FACS analysis showing the cytotoxic effect for CD133 positive glioblastoma multiforme cells of the dendritic cell tumor vaccine according to the comparative example;

FIG. 6 is a set of micrographs showing the cytotoxic effect of the dendritic cell tumor vaccine according to the comparative example and a dendritic cell tumor vaccine according to another embodiment of the present disclosure;

FIG. 7 is a set of micrographs of time-dependent effect of a dendritic cell tumor vaccine cytotoxicity according to another embodiment of the present disclosure; and

FIG. 8 is a line graph of time-dependent effect of a dendritic cell tumor vaccine cytotoxicity according to another embodiment of the present disclosure.

### DETAILED DESCRIPTION

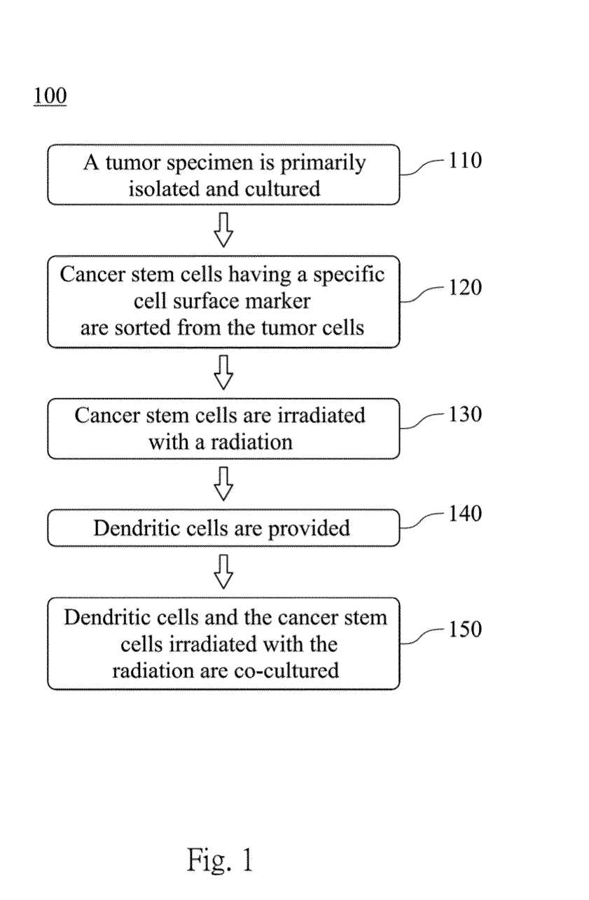

FIG. 1 is a flow diagram showing a method 100 for preparing a dendritic cell tumor vaccine according to one embodiment of the present disclosure. In FIG. 1, the method 100 for preparing the dendritic cell tumor vaccine includes Step 110, Step 120, Step 130, Step 140 and Step 150.

In Step 110, a tumor specimen is primarily isolated and cultured. Specifically, the tumor specimen is collected from a patient, and then primarily isolated and cultured to obtain a plurality of tumor cells. The tumor specimen can be a glioblastoma multiforme.

In Step 120, cancer stem cells having a specific cell surface marker are sorted from the tumor cells. The cancer stem cells can be sorted by using a fluorescence-activated cell sorting (FAGS) or a magnetic cell sorting. Moreover, the cancer stem cells can be CD133 positive cells. In particular, the cancer stem cells can be glioblastoma stem cells. Each of the cancer stem cells can have two specific cell surface markers, and the specific cell surface markers can be CD133 and CD15.

In Step 130, the cancer stem cells are irradiated with a radiation. The dose of the radiation can be 90-110 Gy.

In Step 140, a plurality of dendritic cells are provided. The sources of the dendritic cells can be differentiated from peripheral blood mononucleated cells (PBMCs) collected from the same patient who provides the tumor specimen, thus the dendritic cells are autologous with respect to the cancer stem cells.

In Step 150, the dendritic cells and the cancer stem cells irradiated with the radiation are co-cultured for activating the dendritic cells into cancer-stem-cell-antigen-presenting dendritic cells to obtain the dendritic cell tumor vaccine, wherein the dendritic cell tumor vaccine is a mixture of the cancer-stem-cell-antigen-presenting dendritic cells and the cancer stem cells. That means a cell fusion is not occurred between the cancer-stem-cell-antigen-presenting dendritic cells and the cancer stem cells. In other words, the cancer-stem-cell-antigen-presenting dendritic cells and the cancer stern cells are independent with each other.

The following are descriptions of the specific terms used in the specification:

The term “cancer stem cells (CSCs)” means that the cells are present in a small amount of tumor tissues and have self-renewal ability and differentiation potency. In addition, the cancer stem cells have radioresistance and chemoresistance, which are clinical phenomena of tumor cells that have resistance after radiation therapy and/or chemotherapy. Therefore, the cancer stem cells play an important role in treatment resistance of the cancer patients, tumor recurrence and tumor metastasis.

The term “glioblastoma multiforme (GBM)” means the astrocytomas of the gliomas. GBM is the most aggressive malignant primary brain tumor. A conventional GBM treatment can involve a chemotherapy, a radiation and a surgery. However, the infiltration of the GBM is very high, wherein the infiltration is a migration of cells from their sources to other place. Glial cells in the brain, one of the constituent units of the nervous system, tightly cover axons and provide the functions such as support, nutrition supplement, constant environment maintenance and insulation. Once the glial cells become cancerous, the tumor cells will spread along the axon to the distance because the axon is very long. Surgery could not remove the distant infiltration part of tumor cells and therefore the chemotherapy and/or the radiotherapy is needed for removing the distant infiltration part of tumor cells after the surgery. However, the presence of cancer stem cells having the radioresistance and the chemoresistance causes the high recurrence rate after treatment.

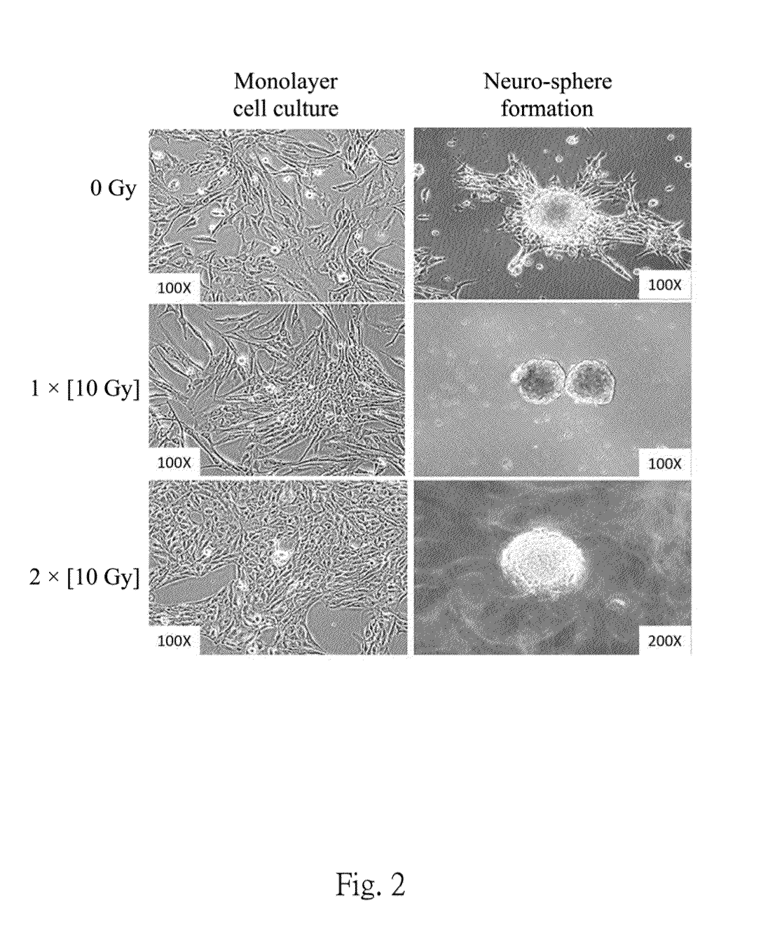

FIG. 2 is micrographs of monolayer cell culture and the neuro-sphere formation of the GBM cells. The GBM cells, which include the GSCs and the GBM primary cells, are cultured in Modified Eagle medium (MEM) with FBS (fetal bovine serum) and stem cell medium respectively. Then the cultured GBM cells are irradiated by a 10-Gy radiation dose from the ¹³⁷Cs source once or twice respectively. As shown in FIG. 2, in the GBM cells cultured in MEM with FBS, both the GSCs and the GBM primary cells adhere to the cell culture dish and the cell morphology is outwardly extending. After irradiating with the radiation, the GSCs begin to aggregate, and the cell morphology of the GBM primary cells is not changed, where still retains the distance between cells. By contrast, in the GBM cells cultured in stern cell medium, the GBM primary cells still adhere to the cell culture dish, but the GSCs form sphere. Moreover, the more times of the radiation are performed; the spherical cell morphology of the GSCs is more obvious. The GBM primary cells are no attachment due to cell death. Thus the results indicate that the different characteristics between the GSCs and the GBM primary cells.

The term “CD133” is a member of pentaspan transmembrane glycoproteins (5-transmembrane, 5-TM). At first CD133 positive cells are recognized in CD34 positive precursor cells, which are isolated from the adult blood, the bone marrow and embryonic stem cells, hence CD133 is considered a marker of hematopoietic stem cells. Afterward CD133 is considered the cell surface marker of the cancer stem cells of the leukemia, the brain cancer, the retinoblastoma, the kidney cancer, the pancreatic cancer, the prostate cancer, the liver cancer, the medulloblastoma and the glioma. The capacities of CD133 positive cells for proliferation and self-renewal are better than normal tumor cells. In addition, the CD133 positive cells have the characteristics of tumorigenicity and sphere formation.

The term “cancer-stem-cell-antigen-presenting dendritic cells” means that dendritic cells that display cancer stem cell antigens bound to major histocompatibility complexes (MHCs) on their surfaces. This process is known as antigen presentation.

Reference will now be made in detail to the present embodiments of the present disclosure, examples of which are illustrated in the accompanying drawings. Wherever possible, the same reference numbers are used in the drawings and the description to refer to the same or like parts.

### EXAMPLES

### Example I

### Isolation and Cell Culture of Glioblastoma Stem Cell

In a first step, GBM specimens are collected. The GBM specimens are washed and chopped into pieces in saline solution, and then disaggregated by using the Papain Dissociation System (WORTHINGTON BIOCHEMICAL) so as to obtain disaggregated GBM cells. In a second step, The disaggregated GBM cells are then resuspended and recovered in stem cell medium (Neurobasal-A medium with B27 supplement, 10 ng/ml EGF (epidermal growth factor) and 10 ng/ml bFGF (basic fibroblast growth factor)) for at least 6 hours to allow re-expression of surface markers. The disaggregated GBM cells are labeled with an allophycocyanin-conjugated or phycoerythrin-conjugated CD133 and CD15 antibodies (MILTENYI BIOTEC. Inc.), and then sorted by fluorescence-activated cell sorting (FACS) or magnetic cell sorting. Thus the glioblastoma stem cells (GSCs) with CD133 cell surface marker and CD15 cell surface marker are sorted from the disaggregated GBM cells. The GSCs are maintained and cultured in the stem cell medium at 37° C. and 5% CO₂ so as to obtain a GSCs population.

### Example II

### Characterization of Glioblastoma Stem Cell

The GSCs population are further confirmed by in vitro test and in viva test. In vitro test, the GSCs population are characterized by neuro-sphere formation assay, gene expression assay and immunoblot analysis. In viva test, the GSCs population are evaluated tumorigenicity by an intracranial xenograft mice model.

### (1) Neuro-Sphere Formation Assay

A neuro-sphere formation assay is represented as the GSCs population on day 0 to confirm the cancer stem cell property. The neuro-sphere formation assay is obtained by plating at a density of 3×10⁸ live cells/per 60-mm dish. After GSCs sphere formation is noted, sphere cells are isolated and plated in 96-well microwell plates in 0.2 ml volumes of the stem cell medium. Final cell dilutions ranged from 200 cells/ well to 1 cell/well in 0.2-ml volumes. Cultures are fed 0.025 ml of the stem cell medium every 2 days until day 7, then calculate a number of cells required to formed at least one neuro-sphere per well. Regression lines were plotted and x-intercept values calculated, which represent the number of cells required to form at least 1 tumor sphere in every well.

### (2) Gene Expression Assay

To confirm the GSCs population in example I are the GSCs, expression levels of OLIGO-2, SOX-2 and CD44 are evaluated by quantative RT-PCR analysis. RNA samples are extracted from the GSCs population by using a RNA isolation kit (QIAGEN). The RNA samples are then subjected to quantative RT-PCR analysis with pairs of primers for GSCs markers to analyze the gene expression of OLIGO-2, SOX-2 and CD44 and primer pair of actin as the internal control.

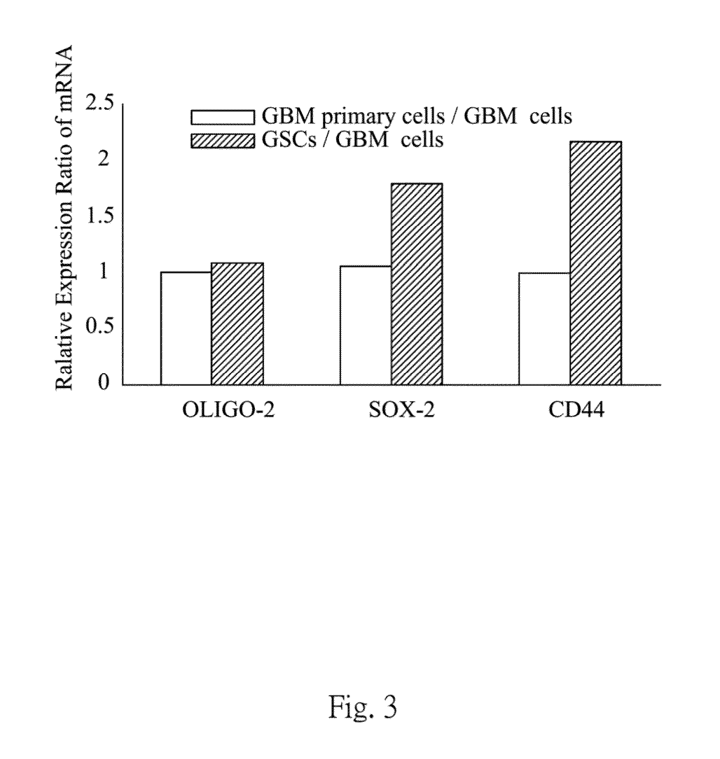

FIG. 3 is a bar chart illustrating relative expression ratio of mRNA of the

GSCs population to the GBM cells according to another embodiment of the present disclosure, which shows the result of example II (2) The GBM cells can be obtained from the first step of example I. The GSCs population can be obtained from the second step of example I. In FIG. 3, mRNA expression levels of OLIGO-2, SOX-2 and CD44 of the GSCs population are significantly higher than mRNA expression levels of OLIGO-2, SOX-2 and CD44 of the GBM cells, wherein expression levels of OLIGO-2, SOX-2 and CD44 are highly expressed in the cancer stem cells. Therefore, the quantative RT-PCR analysis can confirm that the GSCs population are the GSCs.

(3) In Viva Test

The intracranial xenograft mice model includes steps as follows. 3×10⁶ GSCs of the GSCs population in 100 μ1 of PBS (phosphate buffer saline) are subcutaneous injected into the right flank of Scid/bg mice while 1×10⁸ GSCs of the GSCs population in 2 μL of PBS are delivered into the right striatum by stereotactic injection through a glass electrode connected to a Hamilton syringe. The mice are sacrificed at different times between 1 and 10 weeks post-injection. The brain tissues of the sacrificed mice are performed cryostat sections, and then performed Hematoxylin and eosin staining and immunohistochemistry on the 15-μm-thick cryostat sections. The cryostat sections are stained by vimentin, mitochondria, nuclei, Galactocerebroside C, neuronal class III β-tubulin, GFAP, laminin, and Ki67 antibodies to confirm that the GSCs population can form tumors in mice. That is, the GSCs population are the GSCs.

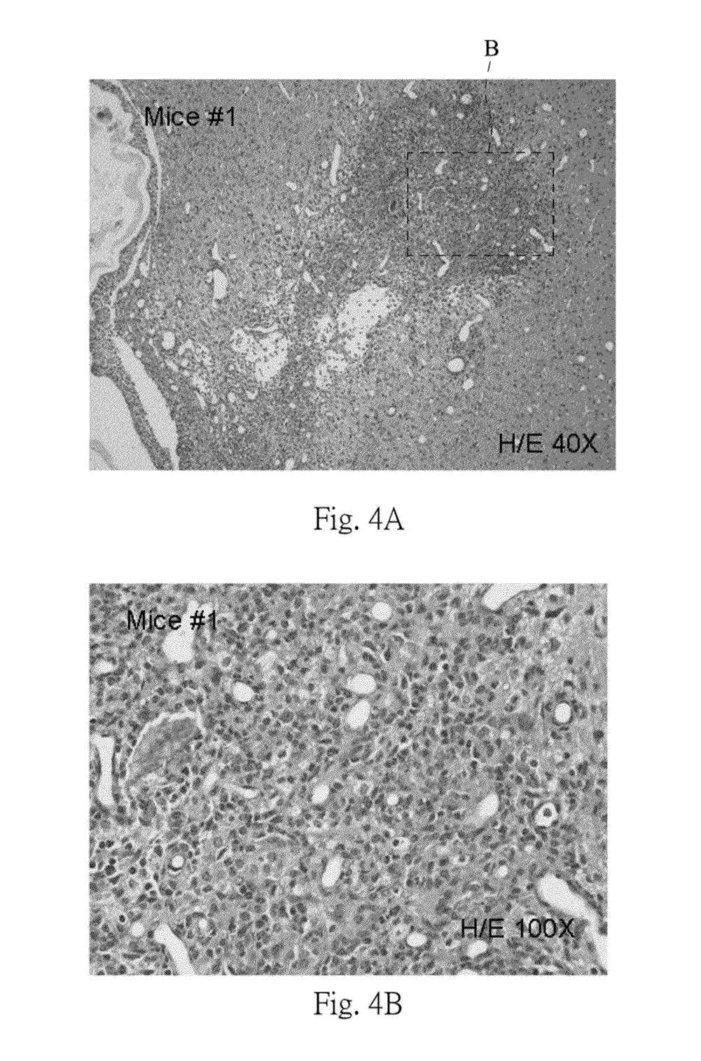

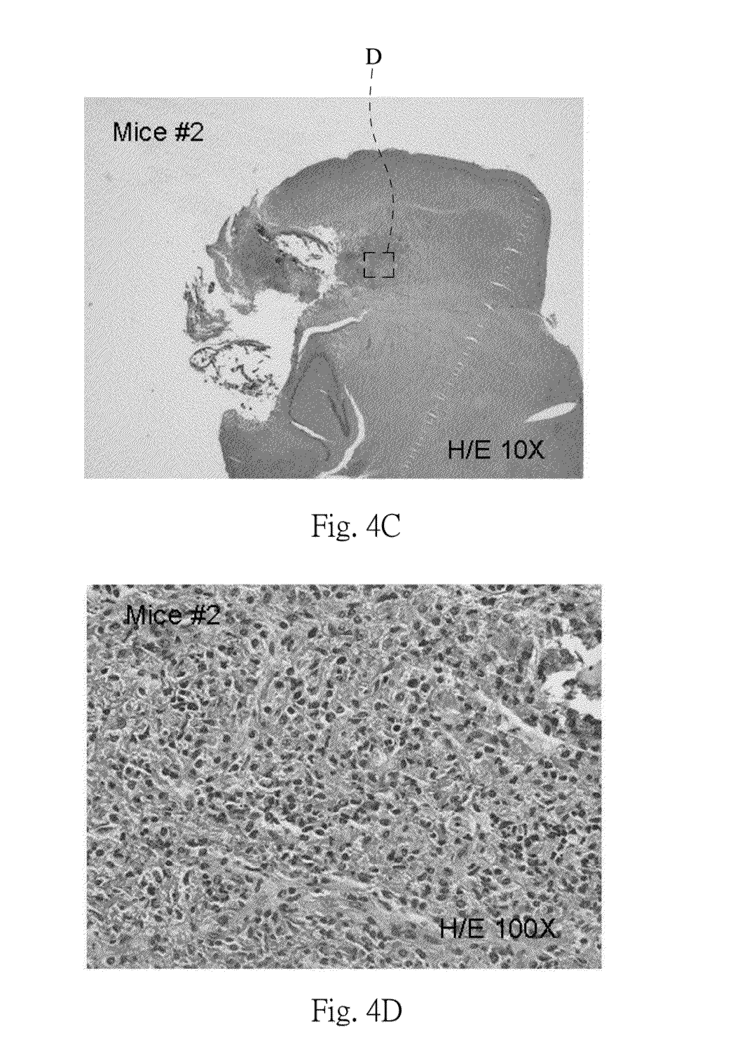

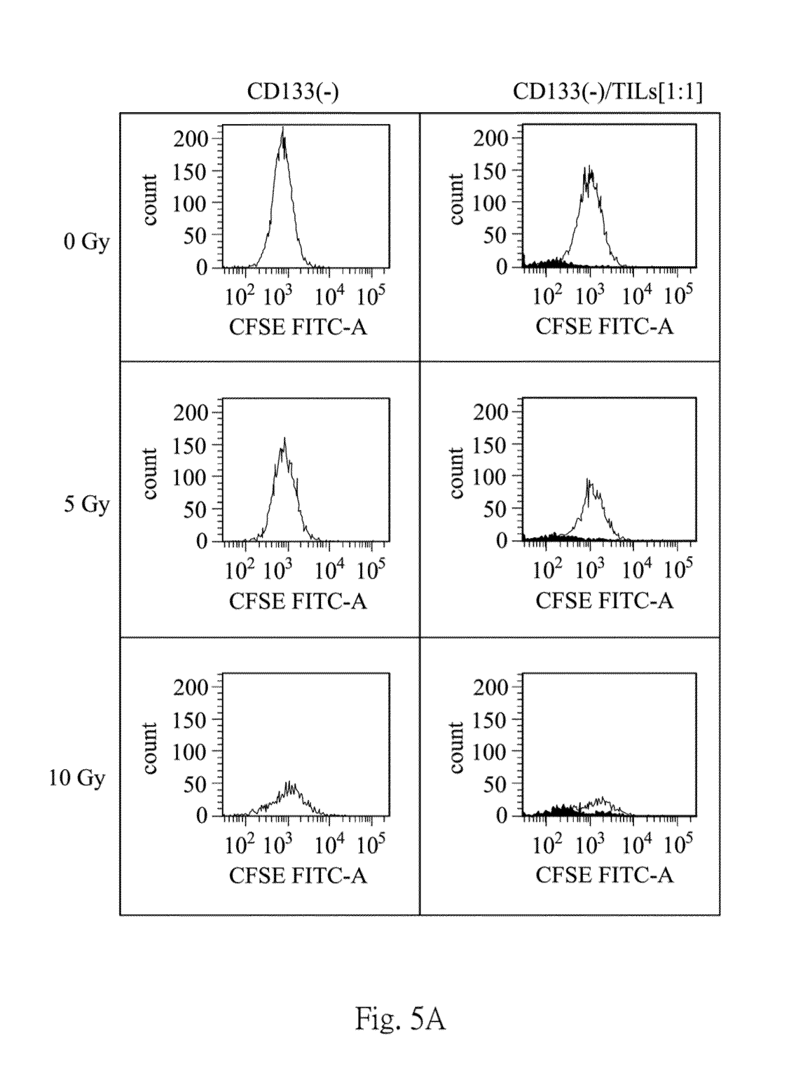

FIG. 4A is a micrograph of the intracranial xenograft mice model of the GSCs by using hematoxylin and eosin staining according to another embodiment of the present disclosure. FIG. 4B is a partially enlarged view of B in FIG. 4A. FIG. 4C is a micrograph of the intracranial xenograft mice model of the GSCs by using hematoxylin and eosin staining according to another embodiment of the present disclosure. FIG. 4D is a partially enlarged view of D in FIG. 4C. In FIGS. 4B and 4D, cancer cells can be recognized by their large size and altered nuclear morphology. The cryostat sections show new tumor growth with neovascular proliferation. Thus the results can confirm that the GSCs population have the characteristics of tumorigenicity.

### Example III

### Preparation of Dendritic Cell

The sources of dendritic cells are differentiated from PBMCs. PBMCs are collected from the same patient who provides the GBM specimens, and then the monocytes are separated through apheresis. The patient's serum (50 to 100 ml) is also collected for in vitro culture use. The isolated monocytes at 2×10 cells/ml are cultured in AIM-V medium (INVITROGEN) with 2% patient's serum. After 2 hours at 37° C., the monocytes adhere to the cell culture dish, and unadhered small lymphocytes are gently washed out by warmed PBS. The residual adhered monocytes are collected and stored in a liquid nitrogen tank at −196° C.

For dendritic cell differentiation, the isolated monocytes are stimulated by granulocyte-macrophage colony-stimulating factor (GM-CSF, BECTON DICKINSON) and interleukin 4 (IL-4, BECTON DICKINSON). The isolated monocytes are cultured in AIM-V culture medium contained 50 ng/ml GMCSF, 1000 U/ml IL-4 and 2% patient's serum at 37° C. and 5% CO₂ for 7 days. Afterwards, dendritic cells are obtained. For dendritic cell confirmation, dendritic cells and lymphocyte cell markers are analyzed by fluorescence-activated cell sorting analysis and flow cytometry with monoclonal antibodies for MHCI and II (BECTON DICKINSON).

### Example IV

### Preparation of Dendritic Cell Tumor Vaccine

The GSCs population are irradiated by a 90-110-Gy radiation dose from a ¹³⁷Cs source. The irradiated GSCs population are then centrifuged and spread into the stem cell medium. The dendritic cells are added to the irradiated GSCs population in a 1:1 ratio and co-cultured under 5% CO₂ for 18 to 24 hours. Finally, the dendritic cells are activated into cancer-stem-cell-antigen-presenting dendritic cells to obtain the dendritic cell tumor vaccine of the present disclosure, wherein the dendritic cell tumor vaccine of the present disclosure is a mixture of thecancer-stem-cell-antigen-presenting dendritic cells and the GSCs population. The dendritic cell tumor vaccine of the present disclosure is diluted by using human serum albumin and divided the cells among 10 tubes. Each tube is contained 2 to 5×10⁷ cells and stored in the liquid nitrogen tank.

### Comparative Examples

### Comparative Example I

### Primarily Cell Culture of Glioblastoma Multiforme Cell

GBM specimens are collected. The GBM specimens are washed and chopped into pieces in Hank balanced salt solution, and then digested by enzymes (40 mg collagenase type IV and 100 U hyaluronidase type V) in Hank balanced salt solution for 3 hours at room temperature. When the GBM specimens become single cell or small tissue fragment suspensions they are filtered by using cell mesh to obtain GBM cells. The solution contained the GBM cells is then added to the patient's serum in MEM and cultured at 37° C. and 5% CO₂, wherein the GBM cells include GBM primary cells and the GSCs population.

### Comparative Example II

### Preparation of Dendritic Cell

The sources of dendritic cells are PBMCs. PBMCs are collected from the same patient who provides the GBM specimens, and then the monocytes are separated through apheresis. The patient's serum (50 to 100 ml) is also collected for in vitro culture use. The isolated monocytes at 2×10⁶ cells/ml are cultured in the AIM-V medium (INVITROGEN) with 2% patient's serum. After 2 hours at 37° C., the monocytes adhere to the cell culture dish, and unadhered small lymphocytes are gently washed out by warmed PBS. The residual adhered monocytes are collected and stored in the liquid nitrogen tank at −196° C. For dendritic cell differentiation, the isolated monocytes are stimulated by GMCSF and IL-4. The isolated monocytes are cultured in the AIM-V culture medium contained 50 ng/ml GMCSF, 1000 U/ml IL-4 and 2% patient's serum at 37° C. and 5% CO₂ for 7 days. Afterwards, dendritic cells are obtained.

### Comparative Example III

### Preparation of Dendritic Cell Tumor Vaccine

The GBM cells are irradiated by the 100-Gy radiation dose from the ¹³⁷Cs source. The irradiated GBM cells are then centrifuged and spread into MEM. The dendritic cells of comparative example II are added to the irradiated GBM cells in a 1:1 ratio and co-cultured under 5% CO₂ for 18 to 24 hours. Finally, the dendritic cells of comparative example II are activated into GBM-antigen-presenting dendritic cells to obtain the dendritic cell tumor vaccine of the comparative example. The dendritic cell tumor vaccine of the comparative example is diluted by using human serum albumin and divided the cells among 10 tubes. Each tube is contained 2 to 5×10⁷ cells and stored in the liquid nitrogen tank.

### Results

### Cytotoxicity of Dendritic Cell Tumor Vaccine to Glioblastoma Stem Cell

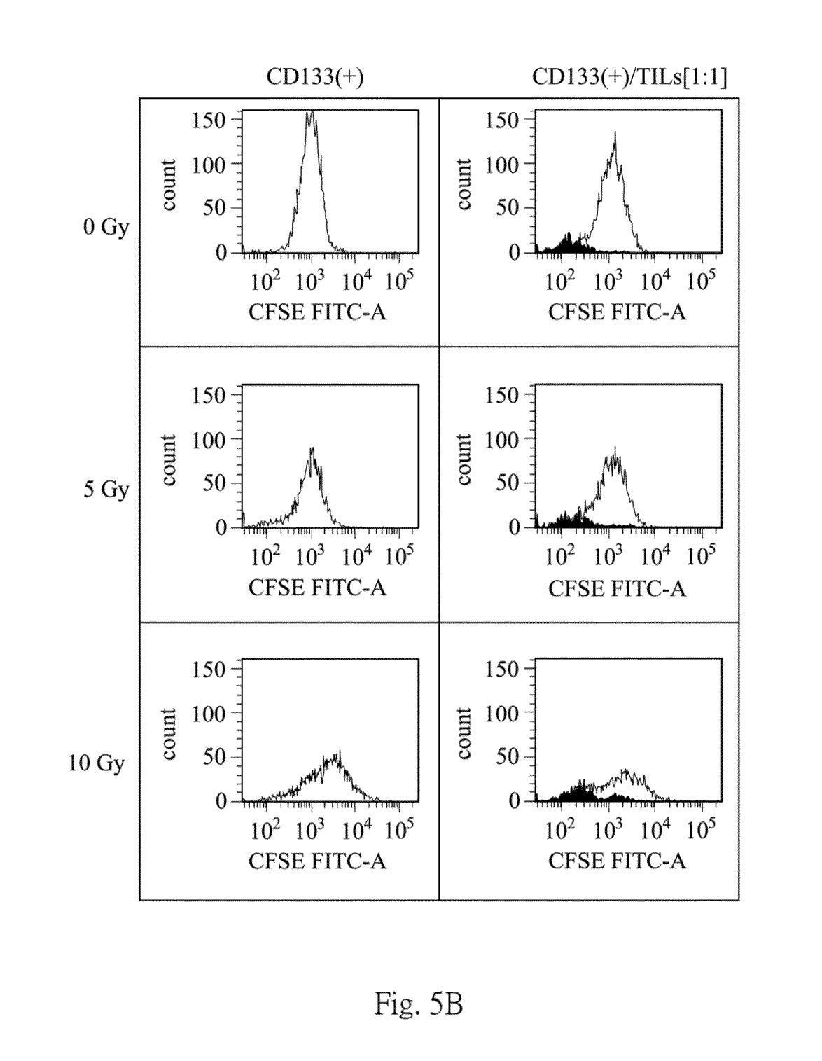

FIG. 5A is a set of histograms of FAGS analysis showing a cytotoxic effect of the dendritic cell tumor vaccine to CD133 negative GBM cells according to the comparative example. FIG. 5B is a set of histograms of FACS analysis showing the cytotoxic effect of the dendritic cell tumor vaccine to CD133 positive GBM cells according to the comparative example. In FIG. 5A and FIG. 5B, the white population of cells represents the GBM primary cells, and the black population of cells represents the GSCs population. The ratio of the dendritic cell tumor vaccine of the comparative example to the GBM cells is 1 to 1, Then the mixture of the dendritic cell tumor vaccine of the comparative example and the GBM cells are irradiated by the 5-Gy and 10-Gy radiation dose from the ¹³⁷Cs source respectively, The results show that the numbers of the GBM primary cells are decreased in both the groups of CD133 negative GBM cells and CD133 positive GBM cells after being irradiated. In particular, the numbers of the GBM primary cells are significant decreased in the group treated the dendritic cell tumor vaccine of the comparative example. However, the numbers of the GSCs are not declined after being irradiated or treated the dendritic cell tumor vaccine. The results indicate that the dendritic cell tumor vaccine of comparative example can not effectively be cytotoxic to the GSCs.

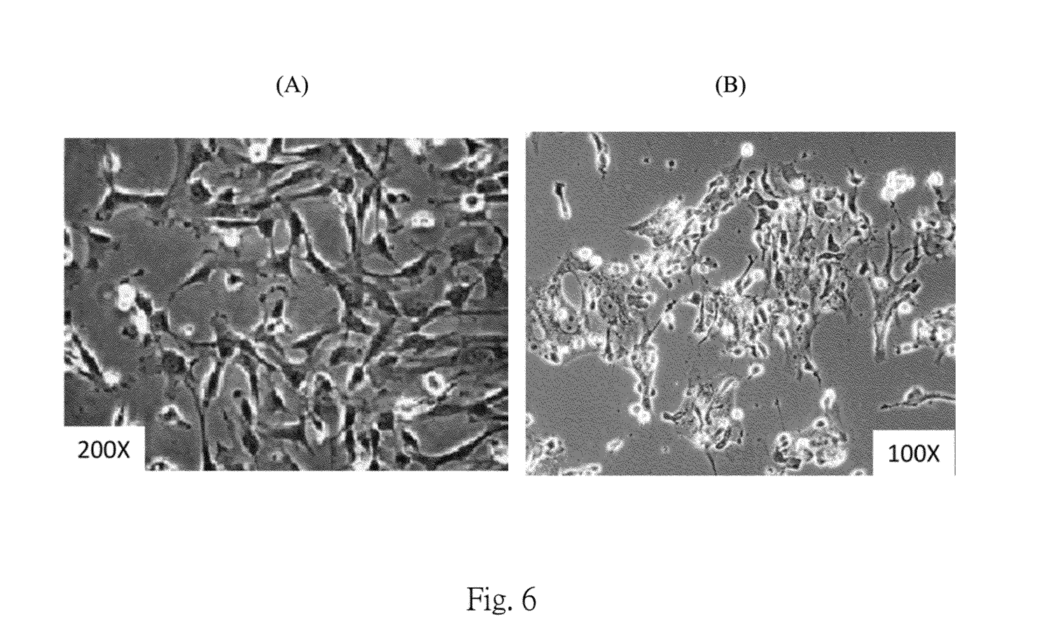

FIG. 6(A) is a micrograph of cytotoxic effect of dendritic cell tumor vaccine according to the comparative example, and FIG. 6(B) is a micrograph of cytotoxic effect of the dendritic cell tumor vaccine according to another embodiment of the present disclosure. In FIG. 6(A), the majority of the GSCs population remain adhered to the cell culture dish and the boundaries between the GSCs population are not affected after being treated the dendritic cell tumor vaccine of the comparative example. However, the GSCs population become larger and their cytoplasm are vacuolated after being treated the dendritic cell tumor vaccine of the present disclosure. Moreover, the GSCs population become aggregated, floated and unhealthy and continuously dead.

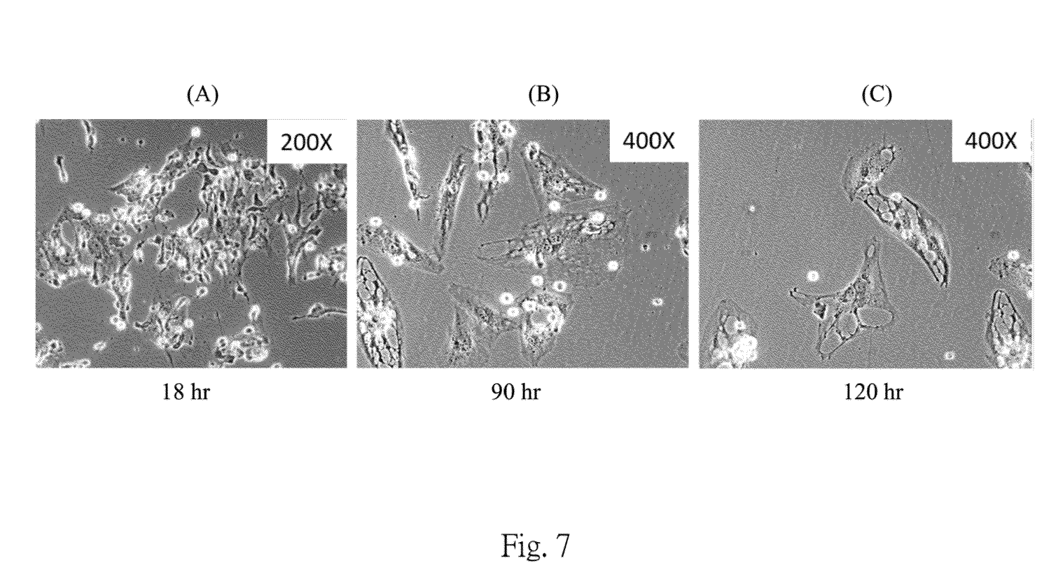

FIG. 7 is a set of micrographs of time-dependent effect of the dendritic cell tumor vaccine cytotoxicity according to another embodiment of the present disclosure. FIG. 7(A), FIG. 7(B) and FIG. 7(C) are the micrographs of the time point of post-treated the dendritic cell tumor vaccine of the present disclosure 18, 90, and 120 hours, respectively. Referring to FIG. 7, the dendritic cell tumor vaccine of the present disclosure has cytotoxicity to the GSCs population at 18 hours, and the cytotoxicity is more obvious at 90 hours and 120 hours. Furthermore, the cytoplasm of the GSCs population is vacuolated, and the GSCs population are gradually dead.

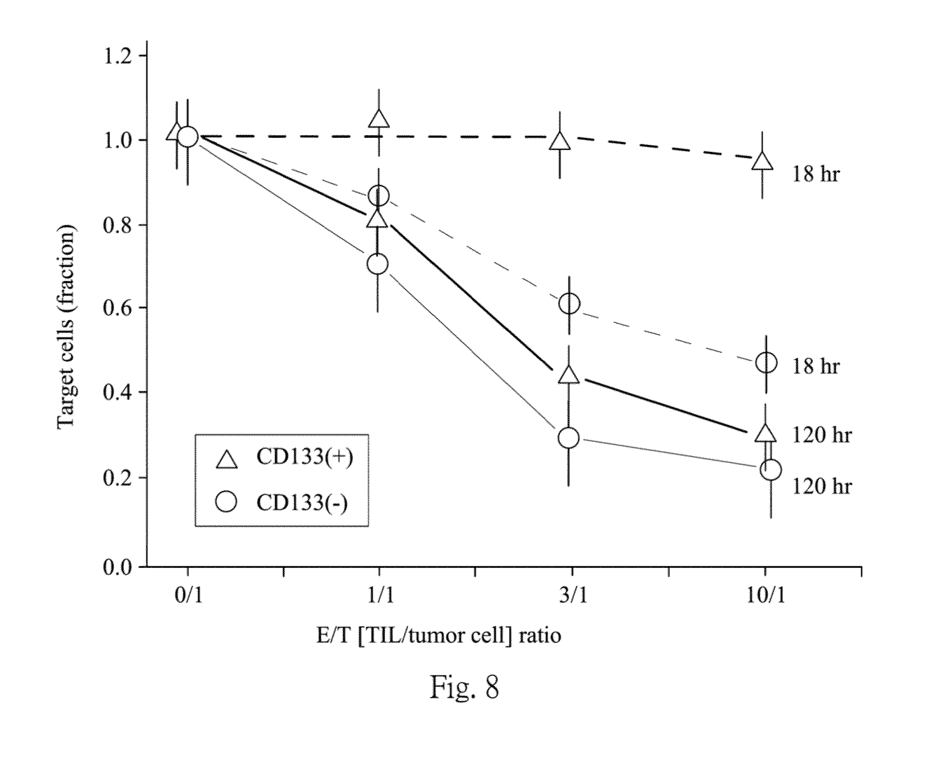

FIG. 8 is a line graph of time-dependent effect of the dendritic cell tumor vaccine cytotoxicity according to another embodiment of the present disclosure. In FIG. 8, the time points are 18 and 120 hours post-treated the dendritic cell tumor vaccine of the present disclosure, and the ratios of the dendritic cell tumor vaccine of the present disclosure to the GBM cells are 1 to 1, 3 to 1 and 10 to 1, respectively. The result in FIG. 8 shows that the dendritic cell tumor vaccine of the present disclosure is not only cytotoxic to the GBM primary cells but also to the GSCs population, especially at 120 hours. In addition, the cytotoxic effect of the GBM primary cells and the GSCs population also is positive correlated with the ratio of the dendritic cell tumor vaccine of the present disclosure to the GBM cells.

The aforementioned method for preparing the dendritic cell tumor vaccine of the present disclosure is not limited to the GBM. It can be used for the preparation of the dendritic cell tumor vaccine for a variety of in vitro tumor tissues. The obtained dendritic cell tumor vaccine has excellent cytotoxicity not only to primary tumor cells but also to the cancer stem cells. Thus, the dendritic cell tumor vaccine of the present disclosure can resolve the problem that the tumor vaccines targeted to the primary tumor cells are not cytotoxic to the cancer stem cells and have high rate of recurrence and metastasis after a treatment, Furthermore, the GSCs and the dendritic cells of the dendritic cell tumor vaccine of the present disclosure are provided from the same patient to obtain an autologous dendritic cell tumor vaccine. The Immunological rejection of the autologous dendritic cell tumor vaccine for the future treatment is low, and the immune comprehensiveness and specificity of the autologous dendritic cell tumor vaccine are advantageous compared with an allogeneic dendritic cell tumor vaccine. Thus, the dendritic cell tumor vaccine of the present disclosure and the preparing method thereof are promising for a cancer treatment.

Although the present disclosure has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present invention without departing from the scope or spirit of the invention. In view of the foregoing, it is intended that the present disclosure cover modifications and variations of this disclosure provided they fall within the scope of the following claims.

What is claimed is:

1. A method of preparing a dendritic cell tumor vaccine, comprising:
(a) primarily isolating and culturing a tumor specimen to obtain a plurality of tumor cells;
(b) sorting cancer stem cells having a specific cell surface marker from the tumor cells;
(c) irradiating the cancer stem cells with a radiation;
(d) providing a plurality of dendritic cells; and
(e) co-culturing the dendritic cells and the cancer stem cells irradiated with the radiation for activating the dendritic cells into cancer-stem-cell-antigen-presenting dendritic cells to obtain the dendritic cell tumor vaccine, wherein the dendritic cell tumor vaccine is a mixture of the cancer-stem-cell-antigen-presenting dendritic cells and the cancer stem cells.

2. The method of preparing the dendritic cell tumor vaccine of claim 1, wherein the tumor specimen is a glioblastoma multiforme.

3. The method of preparing the dendritic cell tumor vaccine of claim 1, wherein the cancer stem cells are glioblastoma stem cells.

4. The method of preparing the dendritic cell tumor vaccine of claim 1, wherein each of the cancer stem cells has two specific cell surface markers, and the specific cell surface markers are CD133 and CD15.

5. The method of preparing the dendritic cell tumor vaccine of claim 1, wherein a dose of the radiation is 90-110 Gy.

6. The method of preparing the dendritic cell tumor vaccine of claim 1, wherein the dendritic cells are autologous with respect to the cancer stem cells.

7. A dendritic cell tumor vaccine, comprising:
a plurality of cancer-stem-cell-antigen-presenting dendritic cells; and
a plurality of cancer stem cells, wherein the cancer stem cells are mixed with the cancer-stem-cell-antigen-presenting dendritic cells.

8. The dendritic cell tumor vaccine of claim 7, wherein the cancer-stem-cell-antigen-presenting dendritic cells are activated from dendritic cells, and the dendritic cells are autologous with respect to the cancer stem cells.

9. The dendritic cell tumor vaccine of claim 7, wherein the cancer stem cells are CD133 positive cells.

10. The dendritic cell tumor vaccine of claim 7, wherein the cancer stem cells are glioblastoma stem cells.
